(19) United States
(12) Patent Application Publication
(10) Pub. No.: US 2016/0235983 A1
(43) Pub. Date: Aug. 18, 2016

(54) SYSTEMS AND METHODS FOR DIRECTED ENERGY CRANIAL THERAPEUTICS

(71) Applicant: NooThera Technologies LLC, Estero, FL (US)

(72) Inventors: Marvin Berman, Plymouth Meeting, PA (US); Michael R. Manning, Langhorne, PA (US); Michael L. Weiner, Estero, FL (US); Warren Woodford, Austin, TX (US)

(21) Appl. No.: 15/018,701

(22) Filed: Feb. 8, 2016

Related U.S. Application Data
(60) Provisional application No. 62/112,838, filed on Feb. 6, 2015.
(60) Provisional application No. 62/112,892, filed on Feb. 6, 2015.
(60) Provisional application No. 62/274,067, filed on Dec. 31, 2015.

(51) Int. Cl. A61N 1/36; A61N 7/00; A61N 2/02; A61N 1/40; A61N 5/06; A61N 2/00

(52) U.S. Cl. CPC A61N 1/36025; A61N 5/0622; A61N 2/006; A61N 2/02; A61N 1/40; A61N 7/00; A61N 2005/0626; A61N 2005/0659; A61N 2005/0662

(57) ABSTRACT

Disclosed is an apparatus for providing cranial therapeutic administration of directed energy. The apparatus may include an energy emission device positionable proximate to a surface of the subject's cranium. Further, the apparatus may include multiple energy portals in communication with any of a variety of suitable sources of directed energy. The multiple energy portals are capable of emitting directed energy directed towards the subject's cranium.

### CROSS-REFERENCE TO RELATED APPLICATION

This application claims priority to and the full benefit of U.S. Provisional Patent Application Ser. No. 62/112,838, filed Feb. 6, 2015, and titled “DEVICE AND PROCESS FOR TREATING DEMENTIA, INCLUDING ALZHEIMER'S, AND OTHER COGNITIVE DISORDERS”, U.S. Provisional Patent Application Ser. No. 62/112,892, filed Feb. 6, 2015, and titled “DEVICE AND PROCESS FOR TREATING DEMENTIA, INCLUDING ALZHEIMERS, AND OTHER COGNITIVE DISORDERS”, and U.S. Provisional Patent Application Ser. No. 62/274,067, filed Dec. 31, 2015 and titled “METHOD AND APPARATUS FOR INDUCING BIOLOGICAL RESPONSE VIA APPLICATION OF ELECTROMAGNETIC RADIATION.” the entire contents of which are incorporated herein by reference.

### FIELD OF THE INVENTION

The present disclosure relates to apparatus for non-invasive inducement of biological responses via administration of directed energy. More specifically, the present disclosure describes focused administration of directed energy to targeted areas of the cranium, for specific periods of time in order to achieve a desired biological response.

### BACKGROUND OF THE INVENTION

Currently, a large percentage of human population is suffering from brain related disorders, both acute and chronic. Existing treatments largely involve administration of pharmacological substances and counseling. However, development and testing of efficacious drugs to treat brain disorders is an extremely lengthy and expensive process. As a result, patients continue to suffer waiting for effective medications. Moreover, the available drugs to treat several mental disorders are not sufficiently effective and produce uncomfortable side effects.

In particular, traditional methods of treating dementia have been varied and in large part ineffective. Generally, dementia patients are treated with systemic chemical treatments that work only to a limited extent. In addition, the prevalent scientific teaching provides that infrared light will not penetrate the brain through the scalp. The result is that the population of people suffering from the symptoms of dementia is increasing.

It is estimated that dementia, affects over 40 million people, worldwide, and with a 10% morbidity another 10% who suffer from progressive memory loss.

### SUMMARY OF THE INVENTION

Disclosed is an apparatus for providing therapeutic administration of directed energy to a subject. In some implementations, the directed energy apparatus may be employed to administer a predetermined directed energy therapy to the cranium of the target subject, such as may be desirable to treat any of a variety of cognitive or other neurological disorders or conditions (e.g. dementia, Alzheimer's, PTSD, TBI, CTE, PTSD, etc. . . . ). The directed energy 2 will penetrate into the brain after passing through the scalp and skull of the subject. The administration of the directed energy into the brain may induce a host of effects or changes on the neurons and other cells in the brain (e.g. glial cells). For example, the effective penetration of directed energy into the cranium of the subject may be used to selectively regulate (up and/or down depending upon the desired outcome) any number of important chemicals and/or cells in the brain, including inducing the generation of adenosine triphosphate (ATP) in the mitochondria of the cells subject to the directed energy therapy.

The apparatus may include an energy emission device that may be positionable proximate to a surface of the subject's cranium. Multiple energy portals are included with the energy emission device. The multiple energy portals may be electrical communication with a source of electromagnetic radiation. Accordingly, the multiple energy portals may be capable of emitting electromagnetic energy directed towards the subject's cranium. Additionally, the apparatus may include a controller which may be in logical communication with the source of electromagnetic radiation. Further, the controller may be operative to cause the source of electromagnetic radiation to provide specific frequencies of electromagnetic energy to at least some of the energy portals. Furthermore, the controller may include a processor and a digital data storage. Additionally, the apparatus may include a biometric measurement device quantifying one or more biometric measurements of the subject. Further, the biometric measurement device may be in logical communication with the controller to provide digital data to the controller. The digital data may be indicative of the one or more biometric measurements. Furthermore, the apparatus may include a source of electrical logic signals in logical communication with the controller. Additionally, the source of electrical logic signals may be capable of causing the controller to receive input from the biometric measurement device and cause the source of electromagnetic radiation to emit energy from a specific selection of energy portals for a predetermined period of time. Further, the specific selection of energy portals may be less than all of the energy portals.

In general, the human brain, in addition to being retrainable, may be considered as an electrical device. Accordingly, the human brain needs a certain amount of power to function properly. If the electrical activity of the human brain goes below a certain level, degeneration and even death may follow. Essentially, the brain needs a certain level of electrical activity or it deteriorates, similar to a poorly divided cell that enters into apoptosis. As a result of inactivity, neurons may undergo degeneration and formation of plaque and related holes may take place. According to the present disclosure, amyloid plaques, which may be viewed as sticky build-up accumulated outside neurons, are not a cause, but rather an outcome of a state of the brain.

The present disclosure describes methods, systems and apparatus to provide electrical stimulation to the brain in order to foster a healthy brain. By providing directed energy in the form of one or more of: infra-red light, near infrared light, millimeter wave light or other controlled bandwidths, a subtle electrical field is generated that penetrates near to the cranium such that the brain receives the directed energy and returns towards its normal state.

In some embodiments, the infrared light emitted by the apparatus may penetrate the brain through the scalp. In addition to the effective penetration of near infrared and other energies such as rTMS, PEMF, etc. may activate vascular endothelial growth factor (VEGF). Further, an increase in ATP which is the fuel of the cell may also be achieved. Accordingly, this may lead to healing of old cells and improvement in other cellular functions, including brain repair. In some embodiments, providing the directed energy at the disclosed frequencies to the cranium, memory loss may be treated even with a short exposure.

However, it may be noted that the frequencies that may be effective treat a normal category of people, may not be suitable for persons suffering from ADHD, or bipolar disorder.

In some embodiments, the apparatus disclosed herein may be configured to enhance one cognitive function and tone down another. For example, the apparatus may be configured to be effective for treating dementia without over-stimulating depression, tinnitus or PTSD.

It may be shown that the use of infrared light at the frequencies disclosed herein may cause the brain to regenerate by rebuilding the neuronal tissue, nerve pads, dendrites and synapses. Similarly, it may be shown that certain frequencies of infrared light applied to bone may cause red bone marrow to start generating stems cells. In fact, there is evidence that infrared light presented on bone can cause heart tissue to regenerate in humans, in situ. In particular, it may be possible to stop degeneration with an electrical signal; induce regeneration with another signal and retrain the brainwaves the loss of coherence provided during degeneration.

One general aspect of the present disclosure includes apparatus for providing trans-cranial energy therapy to a mammalian subject, the apparatus including a directed energy emission device positionable proximate to a surface of a cranium of the subject mammalian subject, the directed energy emission device having a plurality of energy-emitting portals and a source of energy in communication with the plurality of energy-emitting portals. The energy-emitting portals capable of emitting energy in the form of at least one of: visible light, infrared light, near infrared light, electromagnetic fields, ultrasound, and millimeter waves. The emitted energy is directed towards the subject mammalian's cranium via the energy-emitting portals. A controller in logical communication with the source of energy and operative to cause the source of energy to provide a predetermined energy therapy to at least some of the energy portals, wherein the predetermined energy therapy includes delivering at least one type of energy from a specific selection of energy portals for a predetermined period of time with a predetermined energy level.

The controller may include a processor and a digital data storage; a biometric measurement device capable of quantifying one or more biological aspects of the subject mammalian subject. The biometric measurement device in logical communication with the controller at least one of constantly and intermittently to provide digital data to the controller indicative of the one or more measurements quantifying biological aspects of the subject mammalian subject. The biometric measurement device provides digital data to the controller at least one of before, during, and after an administration of the predetermined energy therapy. The biometric measurement device may include one or more of: (i) a neurofeedback system in logical communication with the controller including external stimulation administered to the subject mammalian subject in the form of at least one of visual stimulation, audio stimulation, trans-cranial electrical stimulation, trans-cranial magnetic stimulation, trans-tongue electronical stimulation, and trans-dermal electrical stimulation; (ii) a biofeedback system in logical communication with the source of energy to at least one of continue and modify at least one of the predetermined energy therapy and neurofeedback administered to the subject mammalian subject based on the one or more measurements quantifying biological aspects of the subject mammalian subject; and (iii) a data storage device for storing the one or more measurements quantifying biological aspects from the subject mammalian subject at least one of before, during, and after at least one of the predetermined energy therapy and neurofeedback therapy administered to the subject mammalian subject.

A source of electrical logic signals may be placed in logical communication with the controller and capable of causing the controller to receive input from the biometric measurement device and cause the source of energy to continue delivering the predetermined energy therapy or selectively modify the predetermined energy therapy to the subject mammalian subject based on the input from the biometric measurement device. Modifying the predetermined energy therapy may include at least one of: modifying the selection of the energy portals, modifying a type of energy; modifying a level of energy; modifying a duration of the energy; and modifying a frequency of the energy.

Implementations may include one or more of the following features. The apparatus where the controller is additionally in logical communication with a digital communications network and the source of electrical logic signals includes at least one network access device communicating the electrical logic signals via the digital communications network to deliver the predetermined energy therapy to the subject mammalian subject at a location remote from the controller. The network access device may additionally receive and deliver digital data indicative of feedback from a biometric measurement device to the controller. The controller may be local or at a location remote from the subject mammalian subject. The energy portals may include at least one of: (a) a plurality of light emitting diodes capable of emitting light with a wavelength of between 450 nanometers and 1500 nanometers, (b) a plurality of fiber optic transmission elements each having a distal end for providing luminous communication from a source of light with a wavelength of between 450 nanometers and 1500 nanometers to a respective energy emission portal; (c) one or more ultrasound transducers; (d) one or more millimeter wave transducers; (e) one or more electrodes for delivering at least one of pulsed direct current (dc) and alternating current (ac); (f) one or more electromagnetic coils capable of emitting at least one of pulsed and static electromagnetic fields; and (g) combinational transducers capable of delivering any combination of types of energy.

The multiple energy portals may include a matrix and at least some of the matrix of energy portals may be activated by the controller to emit energy independent of other energy portals. The multiple energy portals may include a matrix with energy portals identifiable via predetermined coordinates selected from one or more coordinate types including Cartesian, polar, number line, cylindrical, spherical, homogenous, curvilinear, orthogonal, skew log-polar, plucker, canonical, parallel, barycentric, trilinear, and any transformation thereof. The controller may activate one or more of the energy portals according to a pattern that may be associated with multiple predetermined coordinates.

The controller may activate a source of energy to temperospatially deliver energy via emitting portals positioned proximate to one or both of a frontal lobe and a parietal lobe, and where the biometric measurement device provides feedback indicative of at least one of stimulation of and reduced activity in, one or both of the frontal lobe and the parietal lobe.

The controller may, in some implementations, identify a front portion and a back portion of the matrix and activate energy portals in a pattern generally beginning at least one of at the front portion and back portion of the matrix and continuing to at least one of the back portion and front portion, respectively. The controller may, in some implementations, identify a portion of the matrix as a left portion and a right portion of the matrix and activates energy portals in a pattern generally beginning with at least one of at the left portion and right portion of the matrix and continuing to at least the right portion and left portion, respectively. may, in some implementations, identify a center portion and a periphery portion of the matrix and activates energy portals in a pattern generally beginning at least one of at the center portion and periphery of the matrix and continuing to at least one of the periphery portion and center portion, respectively, of the matrix and activate energy portals in a stochastic pattern throughout the matrix.

The apparatus may utilize a biometric measurement device that includes an electronic gaming device requiring inputs based upon cognitive analysis and manipulation of a user input mechanism.

The apparatus may utilize a biometric measurement device that includes at least one of: a quantitative electroencephalography (QEEG) apparatus, an electromyography (EMG) apparatus, a thermometer, an electrodermography (EDG) apparatus, a photoplethysmography (ppg) apparatus, an electrocardiogram (ECG) apparatus, a pneumography apparatus, a capnography apparatus, a rheonocephalography (REG) apparatus, and a hemoencephalography (HEG) apparatus.

The apparatus may include a predetermined energy therapy that is capable of regulating production of adenosine triphosphate (ATP) and is selected to at least one of up regulate and down regulate the production of ATP of cells within the cranium of the subject mammalian subject. Application of predetermined energy to regulate ATP production may be selected to have a peak wavelength of at least one of approximately 620 nanometers, approximately 680 nanometers, approximately 760 nanometers, and 820 nanometers. The apparatus may also include predetermined energy to down regulate ATP production and may be selected having a peak wavelength of at least one of approximately 750 nanometers, 870 nanometers, 900 nanometers, and 950 nanometers. The predetermined energy therapy may also be capable of regulating an efficacy and a potency of at least one of pharmaceuticals, over-the-counter supplements, and medications to be administered to or by the subject mammalian subject and is selected to at least one of up regulate and down regulate at least one of the efficacy and potency of at least one of pharmaceuticals, over-the-counter supplements, and medications to be administered to or by the subject mammalian subject.

The apparatus may include predetermined energy therapy capable of regulating at least one of natural dna sequences, synthetic dna sequences, and associated genes, sub-sets and/or pre-cursors of the natural or synthetic dna sequences and is selected to at least one of up regulate and down regulate at least one of natural dna sequences, synthetic dna sequences, and associated genes, sub-sets and/or pre-cursors of the natural or synthetic dna sequences. The apparatus The apparatus may include predetermined energy therapy is at least one of selected: (a) from a pre-existing list of potential energy therapies based on a pre-therapy assessment of the subject mammalian subject with reference to an aggregated database incorporating at least one of pre-therapy assessments of other subject mammalian subjects, energy therapies of other subject mammalian subjects, and results of the energy therapies of other subject mammalian subjects; and (b) by a user of the apparatus based on a pre-therapy assessment of the subject mammalian subject at least one of with and without reference to the aggregated database. The apparatus may include pre-therapy assessment includes at least one of positron emission tomography (pet), computed tomography (ct), single-photo emission computed tomography (spect), blood biomarker testing, and cognitive testing to establish a functional baseline for the subject mammalian subject from which to compare the results of the predetermined energy therapy as determined by the biometric measurement device at least one of during and after the administration of the predetermined energy therapy.

An effectiveness of the energy therapy may be augmented by at least one of physical exercise and mental exercise at least one of before, during and after the energy therapy. A clinical energy system located in a first location may include the energy emission device, the source of energy, the controller and the biometric measurement device. A remote energy system may be located at a second location remote from the first location and include a second energy emission device, a second source of energy, a second controller, and a second biometric measurement device to collectively deliver a predetermined energy therapy as originated by the controller of the clinical energy system to the subject mammalian subject using the remote energy system.

A predetermined energy therapy may be capable of regulating at least one of: an efficacy and a potency of at least one of pharmaceuticals, over-the-counter supplements, and medications to be administered to or by the subject mammalian subject. It may be selected according to a desired function of up and down regulate an efficacy and/or potency of an active agent. An active agent may include one or more of: a pharmaceuticals' an over-the-counter supplements; and a medications to be administered to a subject mammalian subject/In some implementations, a clinical energy system may be capable of: (a) accessing electronic health records of the subject mammalian subject before the an application of the predetermined energy therapy; (b) calculating the an overall quanta of energy of the predetermined energy therapy to be administered to the subject mammalian subject; (c) calculating a degree of modification to the amount of pharmaceutical, over-the-counter supplement and/or medication to be administered to the subject mammalian subject based on the an increase or a decrease in efficacy or potency due to the up or down regulation as a result of the predetermined energy therapy; and (d) communicating the degree of modification to at least one of a prescribing healthcare professional, a pharmacy associated with the subject mammalian subject, and the remote energy system.

### BRIEF DESCRIPTION OF DRAWINGS

FIG. 1 illustrates a system for providing therapeutic administration of directed energy to a subject in accordance with some embodiments.

FIG. 2 illustrates a device for providing therapeutic administration of directed energy to a subject in accordance with some embodiments.

FIG. 3 illustrates a device for providing therapeutic administration of directed energy to a subject utilizing fibre optics in accordance with some embodiments.

FIG. 4 illustrates placement of energy portals in relation to brain lobes for providing therapeutic administration of infrared light to a subject in accordance with some embodiments.

FIG. 5 illustrates a system for providing therapeutic administration of directed energy to a subject in accordance with some embodiments.

FIG. 6 illustrates a block diagram of a biometric measurement apparatus in relation to a subject and a controller.

FIG. 7 illustrates a flow chart of a method for providing therapeutic administration of directed energy to a subject in accordance with some embodiments.

FIG. 8 illustrates a block diagram of a controller configured for providing therapeutic administration of directed energy to a subject in accordance with some embodiments.

FIG. 9 illustrates a network of computers that may be used in an implementation of an automated apparatus for providing therapeutic administration of directed energy to a subject in accordance with some embodiments.

### DETAILED DESCRIPTION OF THE INVENTION

The present invention relates generally to methods, apparatus and systems for beneficial administration of directed energy to the cranium. Directed energy may include an emission of energy positioned to reach a desired location. In some implementations, the directed energy is administered to the surface of the skull and may include any of a variety of suitable forms of directed energy such as visible light, infrared, near infrared, millimeter wave, ultrasound and/or other therapeutic wavelengths or combinations thereof. According to the present invention a pattern of directed energy is administered to the surface of the skull and penetrates the cranium. Biometric measurements are performed on the subject receiving the pattern of directed energy. Specific patterns of directed energy may be based upon results of the biometric measurements.

In some implementations, a controller provides control signals to a source of directed energy administered to the subject. The controller may also receive instruction from a remote operator, such as a health care practitioner located in a remote location.

In various applications, administration of directed energy may be used in treatment of medical conditions such as, for example, one or more of: Traumatic Brain Injury (TBI), Post Traumatic Stress Disorder (PTSD), depression, tinnitus, and various forms of dementia. In particular, the use of the methods and apparatus disclosed herein may result in one or more of: diminishing, stabilizing and reversal of symptoms of dementia, alone or in combination with chemical intervention. Further, symptoms such as loss of memory attributable to Alzheimer's disease and problems resulting from brain damage such as traumatic brain injury (TBI) may also be alleviated. Other conditions that may benefit from the present disclosure include, osteomyelitis, bone virus, sepsis, bleeding, infection and brain damage. Likewise, symptoms associated with a variety of other neurocognitive and disruptive conditions, such as, for example, ADD, ADHD and bipolar disorder may also be relieved through application of the inventions described herein. IN addition, the apparatus and methods disclosed herein may facilitate optimum performance of a healthy brain.

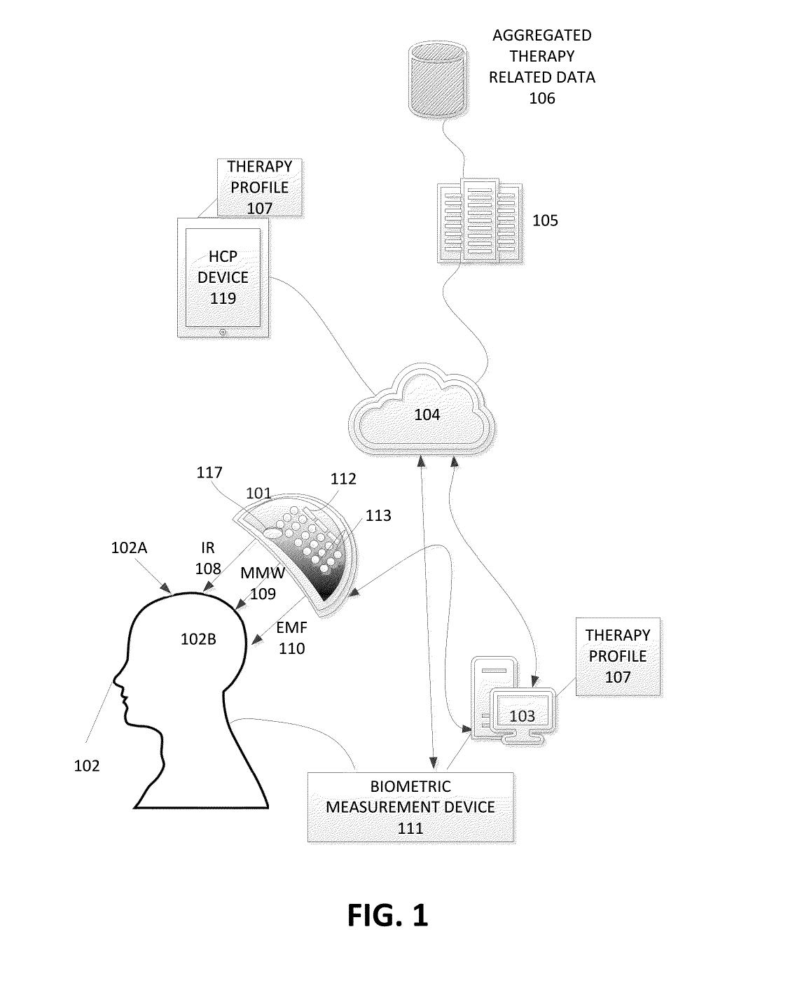

Referring now to FIG. 1, a block diagram illustrates components of a system that is useful for administering trans-cranial directed energy to a skull 102A a subject 102. The directed energy may be administered via a directed energy emission device 101 secured proximate to a surface of a subject's skull 102A. The directed energy emission device 101 will include multiple energy portals 113. In some implementations, the energy portals 113 may include a light emitting diode capable of emitting infrared light with a wavelength of between 450 nanometers and 1500 nanometers. When the directed energy emission device 101 is positioned proximate to the surface of a subject's skull 102A, the energy portals 113 are directed towards the surface of the skull 102A in a fixed position relative to the surface of the skull 102A.

Directed energy may be any suitable form of energy capable of being applied to effect a desired biological response according to the present invention, including but not limited to visible light, infrared light, near infrared light, electrical stimulation (e.g. pulsed DC or AC electrical stimulation), electromagnetic fields (static or pulsed), ultrasound, and millimeter waves. An energy portal 113 is in one or both of: luminous and electrical communication with a source of directed energy 112. The source of directed energy 112 may include, for example, an electromagnetic coil that converts electrical current into a desired wavelength of electromagnetic field and/or a light emitting diode (LED) producing a specific wavelength or band of wavelengths of light (e.g. visible, infrared, near infrared). For example, an energy portal 113 may be in luminous communication with a source of directed energy 112 that is an infrared (“IR”) LED emitting IR directed energy 108. In some embodiments, an LED may be mounted in close proximity to a lens or a lenslet that directs the IR radiation produced by the IR LED to a specific area of the surface of a subject's skull. Accordingly, in some embodiments, a lens 117 may focus IR radiation produced an LED and in other embodiments, a lens 117 may disperse IR radiation emitted by an IR LED. As discussed in more detail below, an energy portal may also include a distal end of a fiber optic conveying directed energy with specific characteristics, such as IR light of a particular bandwidth.

A source of directed energy 112 may also generate and provide directed energy that includes one or more of near infrared light, electromagnetic fields, ultrasound, and millimeter waves. The portals emit the directed energy towards the subject's cranium 102B.

A controller 103 is placed in logical communication with the source of directed energy 112 and operative to cause the source of directed energy 112 to provide a predetermined directed-energy therapy to at least some of the energy portals 113, wherein the predetermined directed energy therapy may include delivering one or more types of directed energy from a specific selection of energy portals 113 for a predetermined period of time with a predetermined energy level. In some embodiments, a specific selection of energy portals 113 specifies less than all of the energy portals 113. As described in more detail below, the controller 103 will include a processor and a source of electrical logic, such as a digital storage device. Electrical logic may include a pattern of electrical signals that may be interpreted in a logical manner.

Further, a biometric measurement device 111 may be included for quantifying one or more biometric measurements of the subject 102. The biometric measurement device 111 may be in logical communication with the controller 103 to provide digital data to the controller 103 indicative of the one or more biometric measurements from the subject 102 at a timing of least one of: before, during and after the administration of the predetermined directed energy therapy.

In some implementations, the biometric measurement device 111 may include an electronic gaming device requiring inputs based upon cognitive analysis and manipulation of a user input mechanism. In some implementations, the biometric measurement device 36 may include an elecroencephalography (EEG, whether standard or quantitative/QEEG) apparatus, an electromyography (EMG) apparatus, a thermometer, an electrodermography (EDG) apparatus, a photoplethysmography (PPG) apparatus, an electrocardiogram (ECG) apparatus, a pneumography apparatus, a capnography apparatus, a rheonocephalography (REG) apparatus, and/or a hemoencephalography (HEG) apparatus. In still other implementations, the biometric measurement device 111 may include any device or apparatus useful for measuring skin tension, electrical conductivity, breath analysis, muscle tension and integrity, cell surface potential, pH, salt, and/or other chemical sensitivity or a neurofeedback system with one or more of: visual stimulation, audio stimulation, trans-cranial electrical stimulation, trans-cranial magnetic stimulation, trans-tongue electronical stimulation, and trans-dermal electrical stimulation

Additionally, a source of directed energy 112 may be placed in logical communication with the controller 103 and capable of causing the controller 103 to receive input from the biometric measurement device 111 and cause the source of directed energy to continue delivering the predetermined directed energy therapy via the energy emission device 101 or selectively modify the predetermined directed energy therapy to the subject 102 based upon the input from the biometric measurement device 111. In some implementations, the predetermined directed energy therapy may be described in a therapy profile 107. Further, modifying the predetermined directed energy therapy may include at least one of modifying the selection of the energy portals 113, the type of directed energy 108-110, the level of directed energy, the duration of the directed energy, and the frequency of the directed energy.

In some implementations, controller 103 is additionally in logical communication with a digital communications network and the source of electrical logic signals is a network access device (described more fully below) with input received under the direction of a healthcare practitioner. The input may include a therapy profile 107. The healthcare practitioner may provide the input generating the logical instruction at a location remote to the location at which the administration of the therapy takes place. The network access device 119 may communicate electrical logic signals via a digital communications network, such as the public Internet and deliver instruction for the predetermined directed energy therapy to be administered the subject 102. Typically the instruction for the predetermined directed energy therapy, which may be embodied in a therapy profile 107, will be delivered to the controller 103. However, it is within the scope of this disclosure that the therapy profile 107 may be communicated via electrical logic directly to the source of directed energy 112.

It is also within the scope of the present disclosure for the network access device 119 to additionally receive and deliver digital data indicative of the one or more biometric measurements. The data may be conveyed via the controller 103. In this manner control of the therapy may be directed from a location remote from the subject 102.

The directed energy emitter array in the example shown includes a plurality of energy portals 113 arranged in a matric type fashion. The of energy portals 113 are spaced evenly apart and are configured to position at least a few of the emitters at strategic places on the user's head. This arrangement is shown by way of example only and it should be noted that the source of directed energy 112 and energy portals 113 may be provided in any suitable number and in any suitable distribution pattern, up to and including a continuous matrix of emitters with no significant space between them. The source of directed energy 112 and energy portals 113 are operable such that any portion of sources of directed energy 112 and energy portals 113 may be activated at any one time, and the combination of the source of directed energy 112 and energy portals 113 may be changed to change the location of the applied directed energy and thereby optimize the therapy. The source of directed energy 112 and energy portals 113 may be configured such that they are all the same, and emit the same type of directed energy. Alternatively, the source of directed energy 112 and energy portals 113 may be configured to emit multiple forms of directed energy. As a further alternative, the plurality of source of directed energy 112 and energy portals 113 may include a mixture of different types of emitters, with each type of emitter configured to emit a different type of directed energy. In this fashion, a user (or therapist) would be able to change the type of directed energy applied to a specific target site. As with previous examples, the apparatus may be in communication with a controller (not shown), either via a wired or wireless connection.

By way of non-limiting example, energy portals may include one or more of: (a) a plurality of light emitting diodes capable of emitting light with a wavelength of between 450 nanometers and 1500 nanometers; (b) a plurality of fiber optic transmission elements each having a distal end for providing luminous communication from a source of light with a wavelength of between 450 nanometers and 1500 nanometers to the respective energy emission portal; (c) one or more ultrasound transducers; (d) one or more millimeter wave transducers; (e) one or more electrodes for delivering at least one of pulsed direct current (DC) and alternating current (AC); (f) one or more electromagnetic coils capable of emitting at least one of pulsed and static electromagnetic fields; and (g) combinational transducers capable of delivering any combination of types of directed energy.

In some implementations, a distributed network 104 may also provide communication to a web server 105 to aggregate therapy related data 106.

Tactical Approach

As described herein, trans-cranial directed energy therapy may include a directed energy emission device positionable proximate to a skull of a cranium providing access to a brain in a cranium and providing low-level NIR stimulation is applied to increase the production of ATP, trigger DNA and RNA expression, and increase the generation of stem cells. Wavelength, power, and direction can be used to target a specific area and activate a desired metabolic process. Neurofeedback techniques, direct neural stimulation, and other sensory stimulation interventions can entrain the use and growth of desired neural pathways. Quantitative EEG analysis can be used to identify targets for neuromodulation and subsequent retraining.

By targeting specific metabolic processes and neural pathways with low-level light therapy (LLLT) and one or more forms of neurostimulation, the present invention achieves one or more of: repair, regeneration, and enhancement of neurological function. Rapid Transcranial Magnetic Stimulation and Transcranial Ultrasound Stimulation are additional sources of stimulation that may contribute to manipulating and controlling synergistic metabolic processes. Clinical trials may test this hypothesis and approach, as well as provide data that can lead to the development of more efficient protocols.

In some implementations, therapeutics may noninvasively assess and remediate dysregulated brain activity, thereby reducing neuropsychiatric symptoms associated with a variety of neuropsychiatric and neurodegenerative disorders. The assessment component utilizes normative data acquired using 19-channel Quantitative EEG (QEEG) neurophysiological measures.

The remediation process may be achieved by delivering a sequence of neuromodulating stimuli, e.g., near infrared light, radio band electromagnetic frequencies and low level, variable frequency, electromagnetic energy.

The system additionally provides for reward conditioning of the EEG rhythms also called neurofeedback training. Neurofeedback utilizing Low Resolution Electromagnetic Tomography (LORETA) analysis permits highly focused training of cortical and subcortical brain regions and neural hubs and networks. Broadly speaking, the clinical goal is to support the repair and retraining of the central nervous system's capacity for optimal performance. Treatment is provided noninvasively using a skull strap, skull cap, helmet-like device or other mechanism to secure a delivery portal of directed energy proximate to an area of treatment. Directed energy may also be used in conjunction with existing standards of care by incorporating a variety of neuromodulation techniques including:

1. reward conditioning of physiological activity,

2. near infrared light stimulation

3. continuous/pulsed transcranial electromagnetic fields

4. focused ultrasound stimulation

5. low energy radio wave-based stimulation tied to ongoing EEG activity

These techniques are seen as both re-energizing and disruptive interventions that can catalyse a reorganization or recalibration of the central nervous system's (CNS's) response to stimulation.

Quantitative assessment of EEG activity provides a real-time measure of brain activation and connectivity performance that has been shown to correlate with specific patterns of attention, motor and behavioural responses. Trauma of various kinds including head injury, childhood physical and emotional abuse and neglect, toxin exposure, etc. are inferred from the patterns of electrical activity in the brain. There is a considerable body of published literature indicating that these events cause changes in the amplitude, range and variability of the EEG's dominant frequency and other measures of neurophysiological activity. Restriction in the range of dominant frequency activity as determined by sampling 256 samples/sec and a moving average displayed at 0.5 sec intervals) can be seen in depressed patients as well as decreased DF variability within the measured frequency range (0.5-38 hz). Norms have also been established for EEG activity by age, gender and handedness.

Applications may leverage both these levels of analysis (DF range & variability) in the construction of integrated intervention protocols and for evaluating their real time effectiveness in normalizing CNS activity. Normalization in this case is defined as optimal conservation of energy, i.e., the CNS's energetic expenditures are only what's required to achieve a given task. The ‘task’ within the intervention process is to maintain a relaxed seated position with eyes open or closed. This approach builds on the notion that efficiency and adaptiveness are highly intercorrelated in all living systems and that increasing the discriminative capacity to make finer distinctions between similarities and differences results in more appropriately adaptive (energy conserving) responses.

According to some embodiments, the apparatus disclosed herein may include software and hardware for acquisition of 19-channels of low impedance EEG and delivery of pulsed or continuous NIR stimulation and pulsed RF band stimulation based on the measured EEG's dominant frequency activity, which is called Neuro-Gen neurofeedback (NGN).

According to another embodiment of this disclosure, a variety of directed energy therapies may be delivered concurrently to treat several different maladies at the same time. This multiple resonance therapeutic (MRT) system may be realized using a standalone dedicated MRT machine or by modifying existing devices that are already being utilized to obtain diagnostic information or perform a different kind of medical treatment (e.g. MRI, CT scan, PET scan, diathermy machine, rTMS, EEG, QEEG, blood pressure analyzer, heart flow output analyzer, oxygen sensors, pressure sensors, and the like) to also include directed energy capability with biofeedback to ensure the best treatment possible.

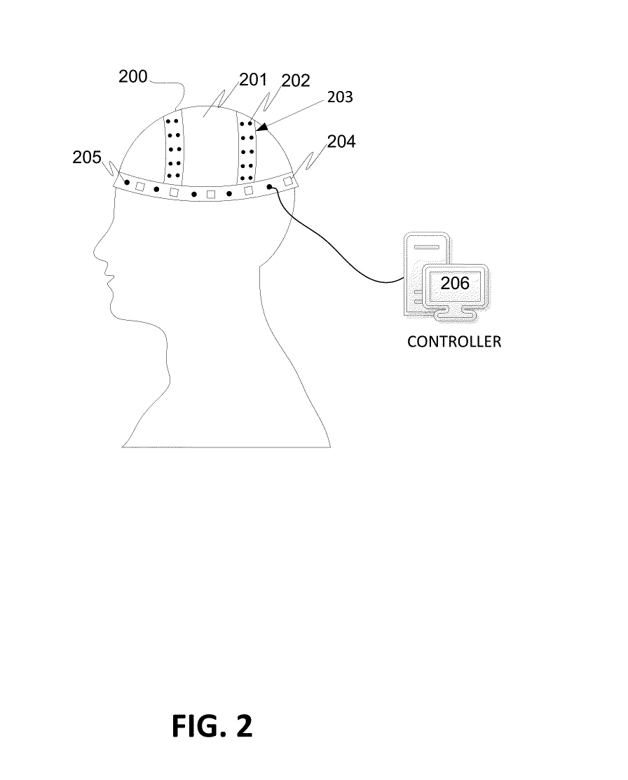

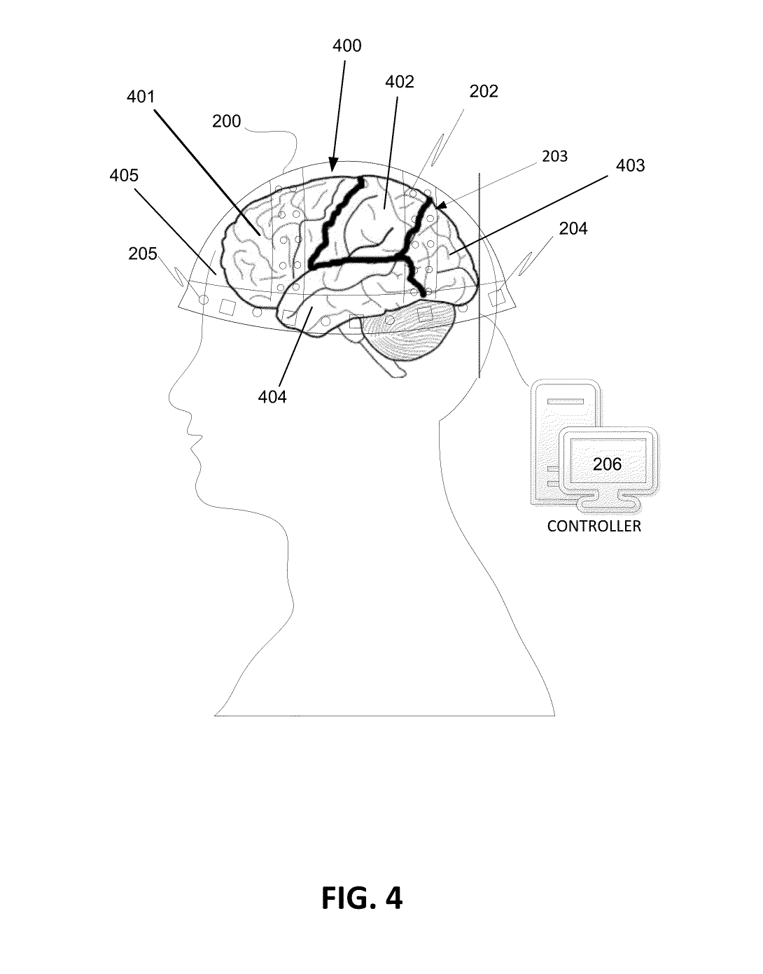

Referring now to FIG. 2, in some implementations, a directed energy emission device 200 may include a harness, straps, skull cap, helmet, cap, band or other support structure suitable for positioning energy portals 202 proximate to a surface area of a subject's skull 201. The energy portals 202 are positioned to direct emitted energy towards a cranium (see FIG. 4) beneath the surface of the area of the skull. In some embodiments, energy portals may be arranged in an array 203 wherein respective energy portals may be selected for transmission of directed energy at a particular instance of time relative to a treatment profile.

Positioning of the energy portals 202 proximate to the subject's skull 201 may if preferable accomplished via fastening techniques allowing for removal with relative ease, while at the same time securing the energy portals 202 relative to portions of the cranium of the subject during administration of a treatment session. Fastening techniques may therefore include, by way of nonlimiting example: Velcro™, elastic constraints, stretchable fabric, a hard shell skull cap, a soft shell skull cap, an adjustable harness, and the like.

In another aspect, a directed energy emission device 200 may include a plurality of energy portals 202 that incorporate a source of directed energy within the portal itself, such as, for example an IR LED that may both generates IR energy and directs the generated IR energy. Other implementations include a plurality of energy portals 202 in one or both of: luminous and electrical communication with a source of directed energy 204. The source of directed energy 204 is capable of emitting energy in the form compatible with a treatment profile. Forms of directed energy may include, by way of non-limiting example, one or more of: near infrared light, infrared light, electromagnetic fields, ultrasound, and millimeter waves.

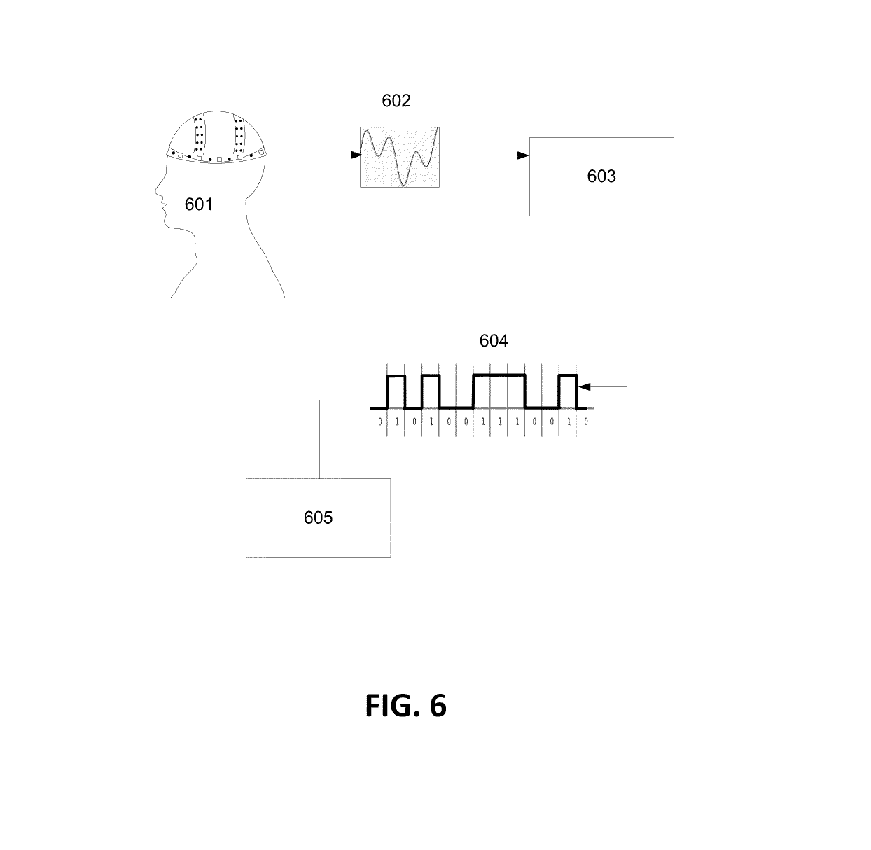

In still another aspect, directed energy emission device 200 may include a biometric measurement device 205 that is operative to perform one or more of measurements quantifying biological aspects the subject. Biometric measurement devices are discussed more fully below in relation to FIG. 6.

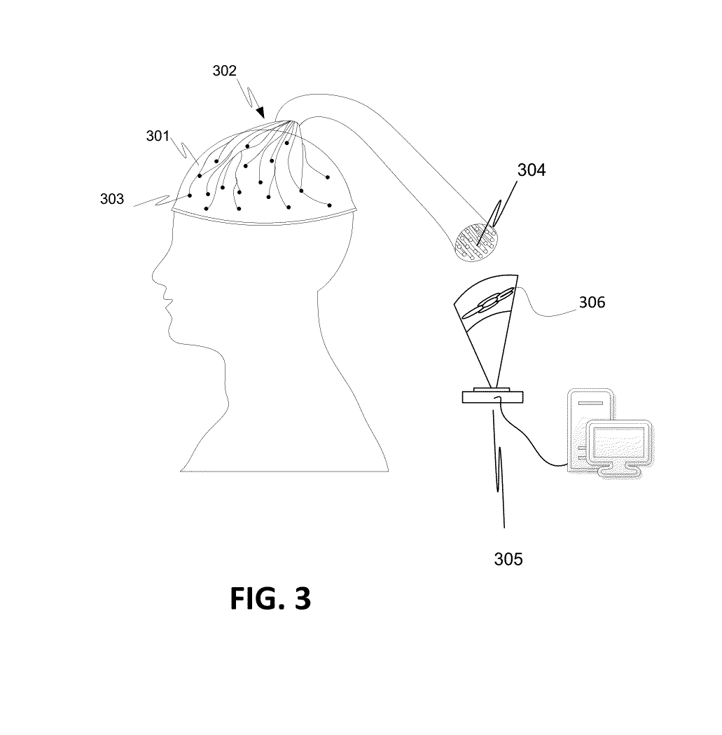

Referring now to FIG. 3, in some embodiments, a directed energy device 301 for administering trans-cranial directed energy incorporates energy portals 303 including a distal end of a fiber optic transmission medium providing luminous communication from a source of directed energy 305. The source of directed energy 305 may include for example, infrared light with a wavelength of between 0.70 μm and 1000 μm. An energy direction device 306 may provide a communication medium from the source of directed energy 305 to respective proximate ends 304 of respective fiber optics 302 and thereby to energy portals 303 via the distal end of the fiber optics 302 terminating in the energy portals 303.

An energy direction device 306 may include, by way of non-limiting example, one or more of: a digital mirror device (DMD), a light wheel, and a light multiplexer or other device capable of providing energy into proximate ends 304 of specific fiber optics. A source of directed energy 305 in luminous communication with the fiber optics 302 may include, for example, one or both of: an infrared light bulb configured to emit energy including a wavelength of between 0.70 μm and 1000 μm and a light emitting diode capable of emitting infrared light with a wavelength of between 0.70 μm and 1000 μm.

Referring now to FIG. 4, a directed energy emission device 200 is illustrated positioning energy portals 202 proximate to a surface area of a subject's skull 201 and over a cranium 405 containing a brain. 300. The energy portals 202 are positioned to direct emitted energy towards a cranium 405 and more specifically towards one or more portions 401-404 of a brain 400 with the cranium 405 and beneath the surface of the area of the subject's skull 201. In some embodiments, energy portals 202 may be arranged in an array 203 wherein respective energy portals 202 may be selected for transmission of directed energy at a particular instance of time and towards a specific area 301-304 of the brain 300. By way of non-limiting example, transmission may be directed towards one or more of: a frontal lobe 401, a parietal lobe 402, an occipital lobe 403 and a temporal lobe 404.

Accordingly, treatment for various symptoms or conditions experienced by the patient may therefore entail directed energy applied to one or more of: treatment of conditions associated with conscious thought, mood changes, planning, control motor skills and social differences may be directed towards a frontal lobe 401; treatment of conditions associated with space, action, integrating sensory information from various senses, and the manipulation of objects, visuospatial processing may be directed towards the Parietal lobe 402; treatment of conditions associated with the sense of sight may be directed towards the Occipital lobe 403; and treatment of conditions associated with the senses of smell and sound, as well as processing of complex stimuli like faces and scenes may be directed towards the Temporal lobe 404.

In a similar manner, areas of the brain within the cranium may be associated with various treatments and patterns of treatments. The controller 206 may be operative to cause the directed energy emission device 200 to apply patterns of directed energy such that energy is directed into the cranium 405 in a pattern including one or more of: beginning with the front of the cranium 405 and transitioning towards a rear portion of the cranium 405; beginning on a right side of the cranium 405 and continuing to a left side of the cranium 405 (or vice versa); beginning in a place generally central to the cranium 405 and expanding radially outward; or other pattern considered to efficacious to achieve a desired result. In some aspects, energy is directed into the cranium in order to reach and treat a specific part of the brain.

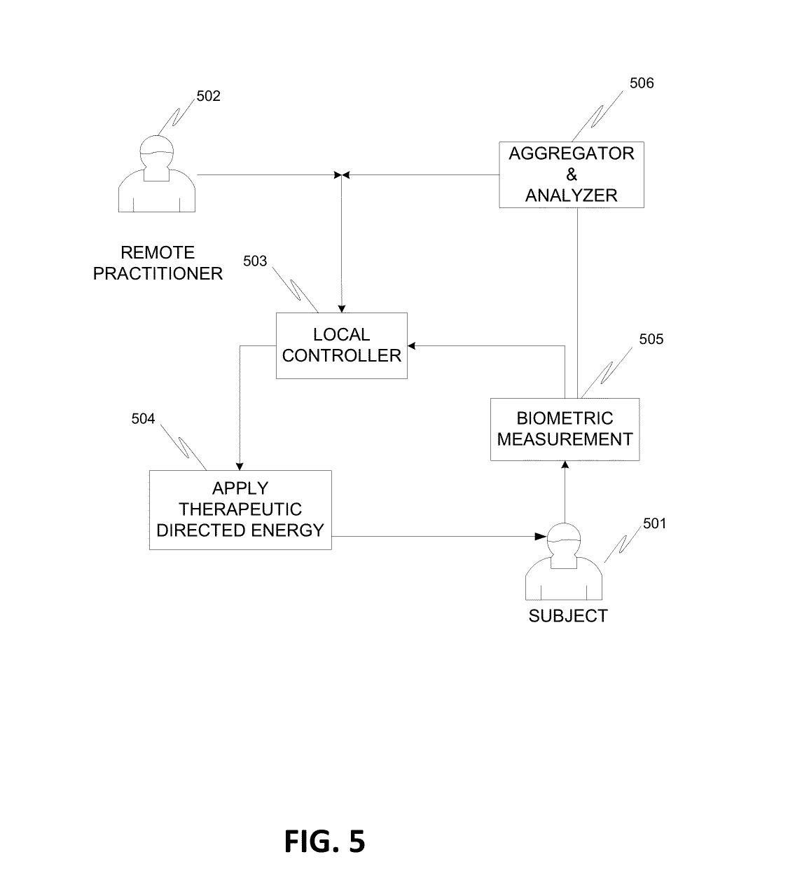

Referring now to FIG. 5 a block diagram illustrates a flow of logic and data that may take place in various implementation of the present disclosure. A local controller 503 may generate electrical signals operative to cause therapeutic directed energy to be applied in an application 504 to a subject 501. One or more biometric measurements 505 may measure biological aspects of the subject 501. The biometric measurements 505 may be made at times that are one or more of: prior to the application 504 of the directed energy, simultaneous with the application 504 of the directed energy; and after the application 504 of the directed energy. Digital data based upon the biometric measurement 505 and in the form of patterns of electrical pulses may be conveyed to one or both of the local controller 503 and a treatment data aggregator and/or analyser 506. In addition the digital data based upon the biometric measurement 505 may correspond with artifacts in a storage device, such as marks in a CD storage, or magnetic charges in magnetic based storage device.

In some implementations a remote practitioner 502 may provide input to remotely operate the local controller 503. Remote operation may be accomplished, for example via a digital communications network, such as the Internet or a cellular network. Digital networks are discussed more fully herein.

Beyond the benefits for trans-cranial applications, directed energy therapy is also useful in a great many non-cranial locations throughout the body. For example, directed energy therapy may help ailing tissue (e.g. organs) if administered so as to up regulate ATP and trigger DNA/RNA expression to thereby speed the recovery or healing process and/or slow a downward progression. Directed energy stimulation has been shown to induce production of opiates, which can reduce pain and speed up tissue regeneration if accompanied with an up regulation of stem cells (per above). Certain forms and levels of directed energy may also affect the circulatory system by causing vasodilation (which helps improve blood flow) or vasoconstriction (which helps decrease blood flow). Directed energy can also be used to reduce inflammation. Wavelength, power, and direction can be used to target a specific area and activate a desired metabolic process.

Directed energy therapy is also capable of regulating the efficacy and/or potency of pharmaceuticals, over-the-counter supplements, and/or medications to be administered to or by the subject by virtue of the ability to selectively up and/or down regulate the various cellular chemicals, stem cells and aspects of cellular function described above. In one embodiment, the directed energy system 2 may be located in a clinical setting (e.g. physician's office), while another directed energy system (not shown but similar to directed energy system 20) may be located in a remote setting (e.g. home of subject) but either remotely monitored or administered or controlled by the clinical system via known or later-developed telemedicine technology or methodology. In this scenario, the clinical directed energy system may be capable of: (a) accessing electronic health records of the subject before the application of the predetermined directed energy therapy; (b) calculating the overall quanta of energy of the predetermined directed energy therapy to be administered to the subject; (c) calculating a degree of modification to the amount of pharmaceutical, over-the-counter supplement and/or medication to be administered to the subject based on the increase or decrease in efficacy or potency due to the up or down regulation as a result of the predetermined directed energy therapy; and (d) communicating the degree of modification to at least one of a prescribing healthcare professional, a pharmacy associated with the subject, and the remote directed energy system.

Neurofeedback techniques, direct neural stimulation, and other sensory stimulation interventions can entrain the use and growth of desired neural pathways. Quantitative EEG analysis can be used to identify targets for neuromodulation and subsequent retraining. Other biofeedback techniques may be used to quantitatively analyze treatment effects in areas other than the brain, for example by measuring skin tension, electrical conductivity, breath analysis, muscle tension and integrity, cell surface potential, pH, salt, and other chemical sensitivity. Verbal feedback from that patient can also be used.

In some embodiments, initially, a clinical diagnosis of a subject may be performed in order to identify a treatable malady. The clinical diagnosis may be based on results of tests conducted using diagnostic devices such as, but not limited to, ultrasound scanners, MRI scanners, CT scanners, EEG readers, PET scan and so on. Further, the clinical diagnosis may also be based on behavioral tests conducted based on questionnaires, interviews, cognitive exercises, physical exercises and so on.

Subsequent to identifying the treatable malady, in some embodiments, one or more regions of the brain associated with the functionality affected by the malady may be identified. For example, in case the clinical diagnosis reveals a condition of dementia, one or more of the frontal lobe, the fronto-temporal lobe and the parietal lobe may be identified for treatment.

In some embodiments, a profiling of the subject may be performed in order to identify one or more of physical characteristics, mental characteristics, emotional characteristics, behavioral characteristics, symptom characteristics, historical treatment characteristics and so on. Further, in some embodiments, a profile of the subject generated based on profiling may be used to query a database in order to identify a treatment plan. Accordingly, the database may include records of various treatment plans indexed according to profiles of patients. For instance, in some embodiments, treatment plans may be indexed according to a brain disorder, a stage of the brain disorder, gender of patient, age of patient and so on. Furthermore, in some embodiments, the profile of the subject may be compared with one or more profiles of other patients who received efficacious treatment. Accordingly, based on a match between a profile of the subject with a profile of an earlier patient, the treatment plan found effective for the earlier patient may be identified as suitable for the subject.

Additionally, in some embodiments, subsequent to retrieving a treatment plan from a database, a medical practitioner may be enabled to modify the treatment plan in order to tailor it for the subject. Alternatively, in some embodiments, based on the clinical diagnosis and/or the profile of the subject, a medical practitioner may devise the treatment plan specific to the subject. However arrived at (e.g. selected, devised, etc. . . . ), once the treatment plan is decided upon as being the one to administer to the subject (e.g. selected or devised) it may thereafter be referred to as the “predetermined directed energy therapy” for the subject.

In general, the treatment plan may include indications regions of the body where therapeutic energy is to be directed. Further, the treatment plan may also include a number of sessions to be administered, duration of each session, a time schedule of the sessions, time sequence of administration of directed energy, and intensities and frequencies of the energy. Furthermore, the treatment plan may also indicate one or more biometric signals to be monitored before, during or after administration of the treatment. Similarly, the treatment plan may also indicate one or more cognitive, behavioral, or physical tests to be administered to the subject before, during or after administration of the treatment. Additionally, the treatment plan may indicate the manner in which current or subsequent administration of directed energy is to be controlled based on the one or more biometric signals and/or results of the cognitive, behavioral, or physical tests.

Referring now to FIG. 6, a biometric measurement device 603 is illustrated in its logical relation between a subject 601 and a processor 605 receiving digital data 604 indicative of a result of a biometric measurement. In general, a subject 601 provides an indication of biological condition 602 of the subject 601. The biometric measurement device 603 receives an indication of the biological condition 602 and converts the indication into digital data 604 representative of the biological condition or aspect. The digital data 604 may in turn be received by a processor 605, such as a controller, that may use the digital data 604 representative of the biological condition or aspect to generate a therapy profile and/or store the digital data 604 representative of the biological condition or aspect for reference by a health care practitioner.

Accordingly, the biometric measurement device 603 is operative to perform one or more of measurements quantifying biological aspects the subject. The biological aspects may include for example empirical data, such as heart rate, QEEG measurements, MRI, sonograms etc, or derived data, such as cognitive response time and accuracy to stimulus. In some embodiments, the biometric measurement device may include an electronic gaming device requiring inputs based upon cognitive analysis and manipulation of a user input mechanism. Further, in some embodiments, the biometric measurement device comprises an EEG apparatus (whether standard or quantitative). In other embodiments, the biometric measurement device may be any that is capable of measuring skin tension, electrical conductivity, breath analysis, muscle tension and integrity, cell surface potential, pH, salt, and other chemical sensitivity.

The biometric measurement device 603 may also be in logical communication with a processor 605 in a controller to provide digital data to the controller indicative of the one or more measurements quantifying biological aspects. The logical communication may include a data feed that is continuous without artificial delay (real time), that is transmitted periodically, or that is transmitted upon command.

Biometric measurements may be conducted on the subject at a timeframe constituting one or more of: before, during and after the administration of the predetermined directed energy therapy. Biometrics may include, by way of non-limiting example, one or more of: QEEG, scans, MRI, cognitive test, reflexive tests, check language of disclosure) ultrasound scanners, MRI scanners, CT scanners, EEG readers, PET scan.

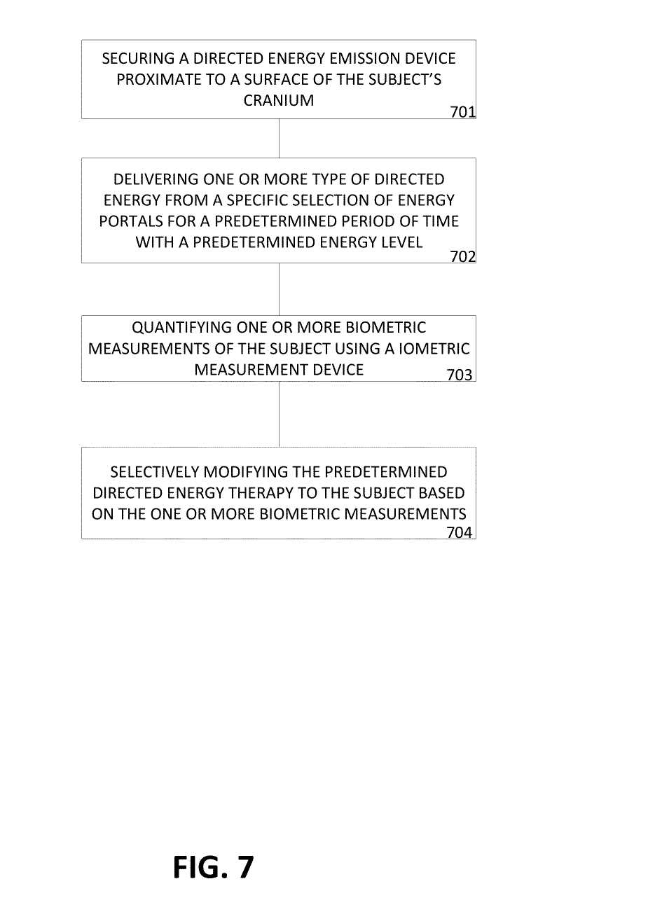

Referring now to FIG. 7, a flow chart of a method for providing therapeutic administration of infrared light to a subject 102 according to some embodiments is illustrated.

In some embodiments, initially, a clinical diagnosis of a subject 102 may be performed in order to identify a brain related disorder. The clinical diagnosis may be based on results of tests conducted using diagnostic devices such as, but not limited to, ultrasound scanners, MRI scanners, CT scanners, EEG readers, PET scan and so on. Further, the clinical diagnosis may also be based on behavioural tests conducted based on questionnaires, interviews, cognitive exercises, physical exercises and so on.

Subsequent to identifying the brain related disorder, in some embodiments, one or more regions of the brain associated with the functionality affected by the brain disorder may be identified. For example, in case the clinical diagnosis reveals a condition of dementia, one or more of the frontal lobe, the fronto-temporal lobe and the parietal lobe may be identified for treatment.

In some embodiments, a profiling of the subject 102 may be performed in order to identify one or more of physical characteristics, mental characteristics, emotional characteristics, behavioural characteristics, symptom characteristics, historical treatment characteristics and so on. Further, in some embodiments, a profile of the subject 102 generated based on profiling may be used to query a database in order to identify a treatment plan. Accordingly, the database may include records of various treatment plans indexed according to profiles of patients. For instance, in some embodiments, treatment plans may be indexed according to a brain disorder, a stage of the brain disorder, gender of patient, age of patient and so on.

Furthermore, in some embodiments, the profile of the subject 102 may be compared with one or more profiles of other patients who received efficacious treatment. Accordingly, based on a match between the profile of the subject 102 with a profile of an earlier patient, the treatment plan found effective for the earlier patient may be identified as suitable for the subject 102.

Additionally, in some embodiments, subsequent to retrieving a treatment plan from a database, a medical practitioner may be enabled to modify the treatment plan in order to tailor it for the subject 102.

Alternatively, in some embodiments, based on the clinical diagnosis and/or the profile of the subject 102, a medical practitioner may devise the treatment plan specific to the subject 102.

In general, the treatment plan may include indications regions of the scalp where therapeutic energy such as infrared light and electric fields are to be directed. Further, the treatment plan may also include a number of sessions to be administered, duration of each session, a time schedule of the sessions, time sequence of administration of infrared light and/or electric fields, intensities and frequencies of infrared light and/or the electric fields. Furthermore, the treatment plan may also indicate one or more biometric signals to be monitored before, during or after administration of the treatment. Similarly, the treatment plan may also indicate one or more cognitive or behavioural tests to be administered to the subject before, during or after administration of the treatment. Additionally, the treatment plan may indicate the manner in which current or subsequent administration of infrared light and/or electric fields is to be controlled based on the one or more biometric signals and/or results of the cognitive or behavioural tests.

Subsequent to identifying the treatment plan, at step 701 a directed energy emission device 101 may be secured proximate to a surface of the subject 102's cranium. The directed energy emission device 101 may include a plurality of energy portals 113 in communication with a source of directed energy 112. The source of directed energy 112 may be capable of emitting energy in the form of at least one of near infrared light, electromagnetic fields, ultrasound, and millimeter waves directed towards the subject 102's cranium.

In some embodiments, the energy portals 113 may include a light emitting diode capable of emitting infrared light with a wavelength of between 0.70 μm and 1000 μm.

Further, in some embodiments, the energy portals 113 may include a distal end of a fiber optic transmission medium providing luminous communication from a source of infrared light with a wavelength of between 0.70 μm and 1000 μm to the respective energy emission portal. Furthermore, the energy emission device 101 may include an infrared light bulb configured to emit energy including a wavelength of between 0.70 μm and 1000 μm.

Additionally, in some embodiments, the energy emission device 101 may include multiple sources of energy generation and at least two of the sources of energy generation may be configured to generate energy in different wavelength bands. Further, the different wavelength bands may include one band of electromagnetic radiation with a wavelength between 0.70 μm and 1000 μm and a second band of electromagnetic radiation with a wavelength greater than 1000 μm.

Further, the method may include a step 702 of delivering one or more type of directed energy from a specific selection of energy portals 113 for a predetermined period of time with a predetermined energy level. Accordingly, a controller 103 in logical communication with the source of directed energy 112, the directed energy may be IR directed energy 108, MMW directed energy 109, EMF directed energy 110 or other wavelength of energy and may be operative to cause the source of directed energy 112 for providing a predetermined directed-energy therapy to at least some of the energy portals 113. Further, the specific selection of energy portals 113 may include less than all of the energy portals 113. The controller 103 may include a processor and a digital data storage.

Additionally, the method may include a step 703 quantifying one or more biometric measurements of the subject 102 using a biometric measurement device. In some embodiments, the biometric measurement device may include an electronic gaming device, such as a gaming device requiring inputs based upon cognitive analysis and manipulation of a user input mechanism. Further, in some embodiments, the biometric measurement device comprises a QEEG apparatus. Additionally, the biometric measurement device may be in logical communication with the controller 103 to provide digital data to the controller 103 indicative of the one or more biometric measurements from the subject 102 at least one of before, during and after the administration of the predetermined directed energy therapy.

Further, the method may include a step 704 of selectively modifying the predetermined directed energy therapy to the subject 102 based on the one or more biometric measurements from the biometric measurement device. The modifying may include at least one of modifying the selection of the energy portals 113, the type of directed energy, the level of directed energy, the duration of the directed energy, and the frequency of the directed energy.

Accordingly, a source of electrical logic signals may be placed in logical communication with the controller 103 and capable of causing the controller 103 to receive input from the biometric measurement device and cause the source of directed energy 112 to continue delivering the predetermined directed energy therapy or selectively modify the predetermined directed energy therapy. Further, in some embodiments, the source of electrical logic signals may be placed in logical communication with the controller 103 including a digital storage storing executable code upon command.

In some embodiments, the multiple energy portals 113 may include a matrix and at least some of the matrix of energy portals 113 may be activated by the controller 103 to emit electromagnetic radiation independent of other energy portals 113.

Additionally, in some embodiments, the multiple energy portals 113 including the matrix may be identifiable via a Cartesian coordinate. Further, the controller 103 may activate one or more of the energy portals 113 according to a pattern that may be associated with multiple Cartesian Coordinates.

In some embodiments, the controller 103 may activate energy portals 113 positioned proximate to one or both of the frontal lobe and the parietal lobe and the biometric measurement device may provide biometric measurements indicative of stimulation of one or both of the frontal lobe and the parietal lobe.

In some embodiments, the controller 103 may activate energy portals 113 positioned proximate to one or both of the frontal lobe and the parietal lobe and the biometric measurement device may provide biometric measurements indicative of reduced activity in one or both of the frontal lobe and the parietal lobe.

In some embodiments, the controller 103 may identify a front portion and a back portion of the matrix and activate energy portals 113 in a pattern generally beginning at the front portion of the matrix and continuing to the back portion of the matrix.

In some embodiments, the controller 103 may identify a left portion and a right portion of the matrix and activate energy portals 113 in a pattern generally beginning at the left portion of the matrix to the right portion of the matrix.

In some embodiments, the controller 103 may identify a center portion and a periphery portion of the matrix and activate energy portals 113 in a pattern generally beginning at the center portion of the matrix to the periphery portion of the matrix.

In some embodiments, the controller 103 may activate energy portals 113 in a stochastic pattern throughout the matrix.

In some embodiments, the biometric measurement device may include an electronic gaming device such as a gaming device requiring inputs based upon cognitive analysis and manipulation of a user input mechanism. In some embodiments, the biometric measurement device comprises a QEEG apparatus.

Furthermore, in some embodiments, the controller 103 may be additionally in logical communication with a digital communications network and the source of electrical logic signals may include a network access device communicating the electrical logic signals via the digital communications network. Further, the network access device may be configured to additionally receive the digital data indicative of the one or more biometric measurements.

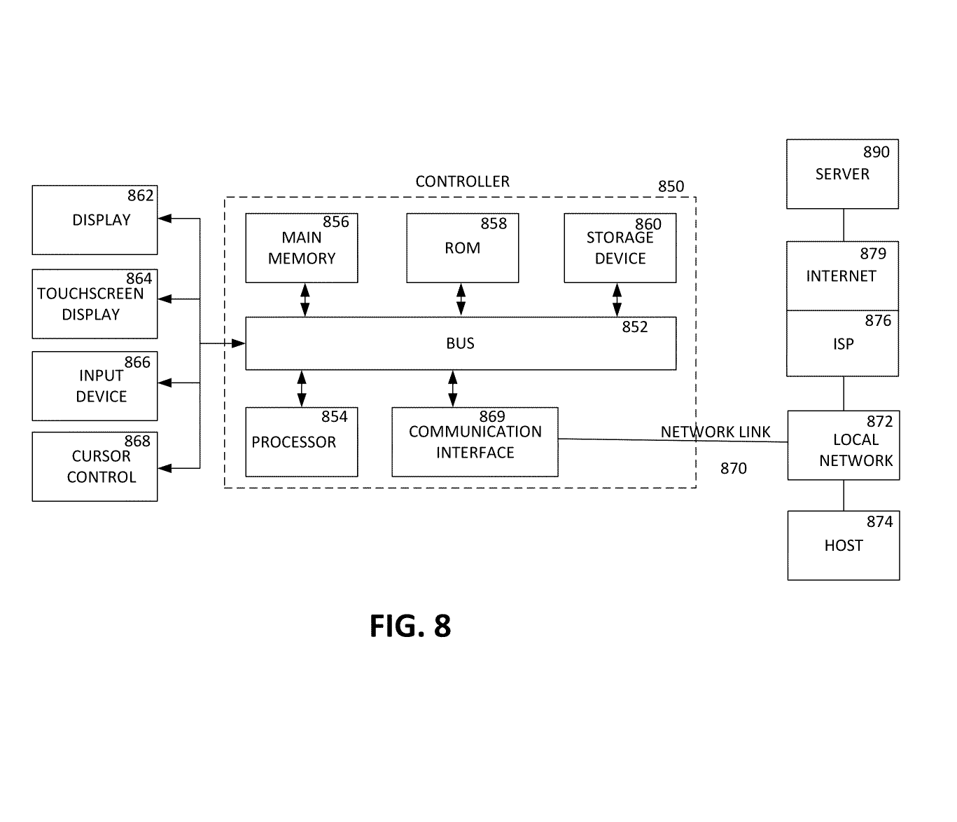

Referring now to FIG. 8, a block diagram of a controller configured for providing therapeutic administration of infrared light to a subject in accordance with some embodiments is illustrated.

Additional aspects of controller hardware which may be included as computer hardware, useful for implementing the present invention may be illustrated as a block diagram that may include a controller 850 upon which an embodiment of the invention may be implemented. Controller 850 may include a bus 852 or other communication mechanism for communicating information, and a processor 854 coupled with bus 852 for processing information.

Controller 850 may also include a main memory 856, such as a random access memory (RAM) or other dynamic storage device, coupled to bus 852 for storing information and instructions to be executed by processor 854. Main memory 856 may also be used for storing temporary variables or other intermediate information during execution of instructions to be executed by processor 854. Controller 850 may further include a read only memory (ROM) 858 or other static storage device 860.

Controller 850 may be coupled via bus 852 to a display 862, such as a liquid crystal display (LCD), for displaying information to a computer user. An input device 866, including alphanumeric and other keys, or modes of input, such as, for example, a microphone and a radio frequency device such as Bluetooth, may be coupled to bus 852 for communicating information and command selections to processor 854. Another type of user input device may be a cursor control 868, such as a mouse, a trackball, a touchpad, touchscreen 864, or cursor direction keys for communicating direction information and command selections to processor 854 and for controlling cursor movement on display 862. This input device may typically have two or three degrees of freedom in two axes, a first axis (e.g., x) and a second axis (e.g., y), that allows the device to specify positions in a plane or stereo cameras that process and provide a third axis of input.

Some embodiments of the invention may be related to the use of controller 850 for setting operational parameters. According to one embodiment of the invention, control parameters may be defined and managed by controller 850 in response to processor 854 executing one or more sequences of one or more instructions contained in main memory 856. Such instructions may be read into main memory 856 from another computer-readable medium, such as storage device 860. Execution of the sequences of instructions contained in main memory 856 causes processor 854 to perform the process steps described herein. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein may refer to any medium that participates in providing instructions to processor 854 for execution. Such a medium may take many forms, including but not limited to, non-volatile media, volatile media, and transmission media. Non-volatile media includes, for example, solid state devices (SSD) or magnetic disks, such as storage device 860. Volatile media may include dynamic memory, such as main memory 856. Transmission media includes coaxial cables, copper wire and fiber optics, including the wires that comprise bus 852. Transmission media may also take the form of infrared and radio frequency transmissions, acoustic or light waves, such as those generated during radio wave and infrared data communications.

Common forms of computer-readable media may include, for example, a memory stick, hard disk or any other magnetic medium, an optical medium, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 854 for execution. For example, the instructions may initially be carried on a magnetic disk of a remote computer. The remote computer can load the instructions into its dynamic memory and send the instructions over a distributed network such as the Internet. A communication device may receive the data on the telephone line and use an infrared transmitter to convert the data to an infrared signal. An infrared detector may receive the data carried in the infrared signal and appropriate circuitry can place the data on bus 852. Bus 852 may carry the data, or otherwise be in logical communication to the main memory 856, from which processor 854 retrieves and executes the instructions. The instructions received by main memory 856 may optionally be stored on storage device 860 either before or after execution by processor 854.

Controller 850 may also include a communication interface 869 coupled to bus 852. Communication interface 869 provides a two-way data communication coupling to a network link 870 that may be connected to a local network 872. For example, communication interface 869 may operate according to the internet protocol. As another example, communication interface 869 may be a local area network (LAN) card a data communication connection to a compatible LAN.

Network link 870 may typically provide data communication through one or more networks to other data devices. For example, network link 870 may provide a connection through local network 872 to a host computer 874 or to data equipment operated by an Internet Service Provider (ISP) 876. ISP 876 in turn may provide data communication services through the worldwide packet data communication network now commonly referred to as the “Internet” 879. Local network 872 and Internet 879 may both use electrical, electromagnetic or optical signals that carry digital data streams. The signals may be transmitted through the various networks and the signals on the network link 870 and through communication interface 869, which carry the digital data to and from controller 850 are exemplary forms of carrier waves transporting the information.

In some embodiments, Controller 850 may send messages and receive data, including program code, through the network(s), network link 870 and communication interface 869. In the Internet example, a server 890 might transmit a requested code for an application program through Internet 879, ISP 876, local network 872 and communication interface 869.

Processor 854 may execute the received code as it is received, and/or stored in storage device 860, or other non-volatile storage for later execution. Some exemplary controllers 850 may include a personal digital assistant, a mobile phone, a smart phone, a tablet, a netbook, a notebook computer, a laptop computer, a terminal, a kiosk or other type of automated apparatus. Additional exemplary devices may include any device with a processor executing programmable commands to accomplish the steps described herein.

A controller may include one or more of: personal computers, laptops, pad devices, mobile phone devices and workstations located locally or at remote locations, but in communication with the controller. System apparatus may include digital electronic circuitry included within computer hardware, firmware, software, or in combinations thereof. Additionally, aspects of the invention may be implemented manually.

Apparatus of the invention may be implemented in a computer program product tangibly embodied in a machine-readable storage device for execution by a programmable processor and method actions can be performed by a programmable processor executing a program of instructions to perform functions of the invention by operating on input data and generating output. The present invention may be implemented advantageously in one or more computer programs that are executable on a programmable system including at least one programmable processor coupled to receive data and instructions from, and to transmit data and instructions to, a data storage system, at least one input device, and at least one output device. Each computer program may be implemented in a high-level procedural or object oriented programming language, or in assembly or machine language if desired, and in any case, the language can be a compiled or interpreted language. Suitable processors may include, by way of example, both general and special purpose microprocessors.

Generally, a processor may receive instructions and data from a read-only memory and/or a random access memory. Generally, a computer may include one or more mass storage devices for storing data files; such devices include Solid State Disk (SSD), magnetic disks, such as internal hard disks and removable disks magneto-optical disks and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD ROM disks may be included. Any of the foregoing may be supplemented by, or incorporated in, ASICs (application-specific integrated circuits).

The specific hardware configuration used may not be particularly critical, as long as the processing power is adequate in terms of memory, information updating, order execution, redemption and issuance. The controlling logic may use a language and compiler consistent with that on a CPU included in the controller. These selections may be set according to per se well-known conventions in the software community.

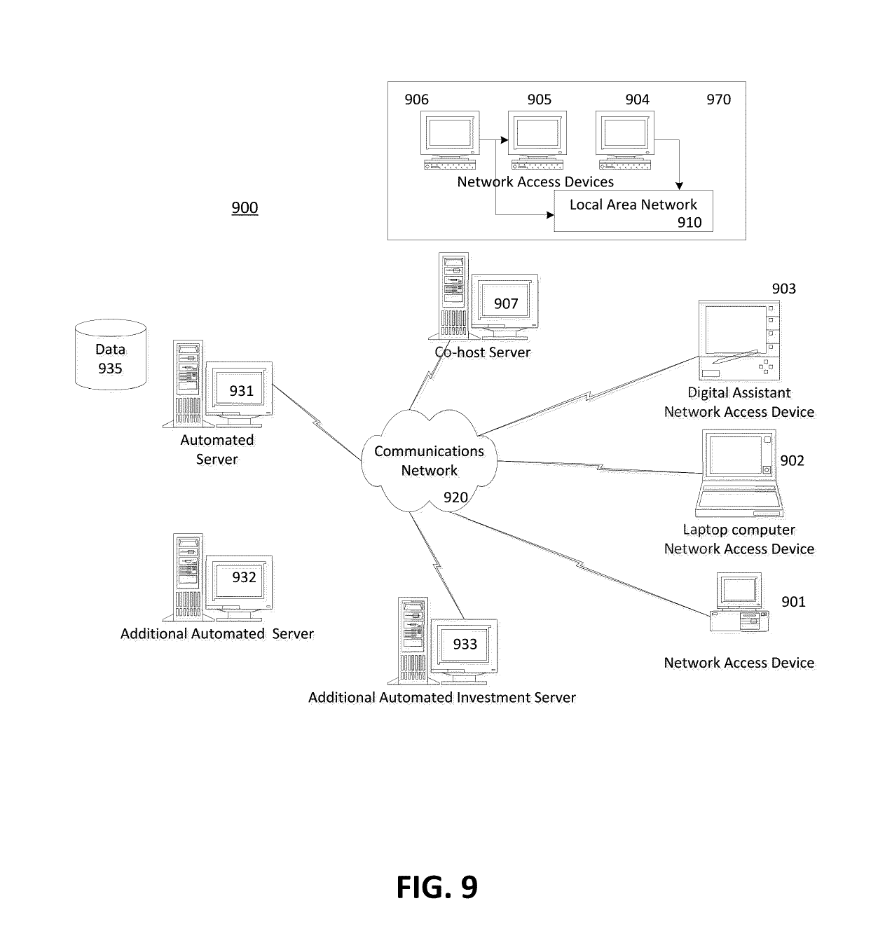

FIG. 9 shows a network 900 of computers that may be used in an implementation of an automated apparatus for providing therapeutic administration of infrared light to a subject in accordance with some embodiments. The network 900 includes an automated server 931 or other host system and client computers 901-903. In some embodiments, the network 900 may include a Digital Communications Network (DCN 920). A DCN 920, as used herein, includes a medium for communicating digital data. Exemplary DCNs 920 include, but are not limited to: the public Internet, a cellular network, and a virtual private network.

Each of the client computers may include a processor, memory, a user input device, such as a keyboard and/or mouse, and a user output device, such as a video display and/or printer. The client computers 901-903 may communicate with the technology server 931 to obtain data stored at the technology server 931.

A client computer 901-903 may interact with the System's Server 931 as if the Server 931 was a single entity in the network 900. However, the Server 931 may include multiple processing and database sub-systems, such as cooperative or redundant processing and/or database servers 941-944, that may be geographically dispersed throughout the network 900. In addition, there may be more than one occurrence of a host server 932. A local server 907 may be a proxy server or a caching server. Server 907 may also be a co-host server that may provide services in conjunction with treatments. A Server 931 includes one or more databases 945 which may store data.

A user may access the Server 931 using client software executed at the user's network access device such as client computers 901-903. The client software may include a generic hypertext markup language (HTML) browser, such as Google Chrome, Firefox, Safari, Opera or Microsoft Internet Explorer, (a “WEB browser”). The client software may also be a proprietary browser, and/or other host access software. In some cases, an executable program, such as a Java™ program, may be downloaded from the technology server 931 to the client computer and executed at the client computer.

The invention may be implemented in digital electronic circuitry, or in computer hardware, firmware, software, or in combinations of them. Apparatus of the invention may be implemented in a computer program product tangibly embodied in a machine-readable storage device for execution by a programmable processor; and method steps of the invention may be performed by a programmable processor executing a program of instructions to perform functions of the invention by operating on input data and generating output. Preferably the invention will be implemented in one or more computer programs that are executable on a programmable system including at least one programmable processor coupled to receive data and instructions from, and to transmit data and instructions to, a data storage system, at least one input device, and at least one output device. Each computer program may be implemented in a high-level procedural or object-oriented programming language, or in assembly or machine language if desired; and in any case, the language may be a compiled or interpreted language. Suitable processors include, by way of example, both general and special purpose microprocessors.

Automated apparatus included in, or connected to in an automated system may be connected to each other by one or more network interconnection technologies. For example WiFi networks, data lines and cellular networks. Functions associated with separate processing and database servers in the server 931 may be integrated into a single server system or may be partitioned among servers and database systems that are distributed over a wide geographic area.

By way of non-limiting example, client computers 901-903 may comprise a personal computer executing an operating system such as Microsoft Windows™, UNIX™, Linux or an Apple operating system, as well as software applications, such as a web browser. Client computers 901-903 may also be terminal devices, or a mobile phone WEB access device, a tablet that adheres to a point-to-point or network communication protocol such as the Internet protocol. Other examples may include TV WEB browsers, terminals and wiss access devices (such as an Android device). A client computer or other Web Access Device may include a processor, RAM and/or ROM memory, a display capability, an input device and hard disk or other relatively permanent storage. Interactive graphical user interfaces specifically related to information generated as a result of the workings of the financial structuring system may include any information contained within databases relating to the financial structure, or derivative of such information.

Studies:

The inventors have discovered therapeutic use of infrared and conducted a double blind, placebo controlled study along with a colleague. The results of the study were presented to the officials in charge of Alzheimer's at the National Institute of Health. Berman discovered that, by combining the infrared energy to the brain for approximately six minutes a day (at the optimal frequency and energy discovered for his device), and then applying the neurofeedback technology using Neuroguide software from Applied Neuroscience out of St. Petersburg, Fla., for two to three times per week, he could get about a 50% increase over and above the infrared by adding brainwave retraining to the recovering brain.

In another aspect, the inventors have discovered that a much lower electric field, almost imperceptible, at the right frequency, outside the brain and without breaking the skin, is enough to provide efficacious effects.

In one instance, the inventor of the present disclosure has discovered the therapeutic effectiveness of infrared light. Daily application of infrared light from a 660 nanometer handheld lamp to areas of scalp was found to be effective in treating dementia. In addition to the infrared light, it was also found that brain retraining performed three times a week from a certified biofeedback expert also improved effectiveness.

Studies have shown that as a result of applying infrared radiation and/or electric fields, the brain can be transitioned significantly outside two standard deviations from a normative database.

According to one study conducted by Berman, a patient was subjected to an infrared light therapy combined with Neuroguide neurobiofeedback. Initially, the QEEG map of the patient indicated significant left frontal damage from a TBI. The therapy included two engagements of brain training using Neuroguide. In addition, three treatments of 6 minutes each with a Dougal helmet using 1,200 LEDs tuned to around 1070 nanometers was also used on the patient.

Prior to the treatment, the patient exhibited noticeable delay in word and synonym retrieval, and difficulty using Smartphones. However, following treatment, the patient reported some easing of memory loss for approximately two weeks.

Accordingly, use of the Dougal helmet in conjunction with the Neuroguide software has produced significant improvement in cognition, as tracked by Neuroguide.

Further, Berman also reported that the infrared light therapy generated significant stabilization of the degeneration, for as long as fifteen months with one patient.

Berman noticed that another increase was achieved with his treatment of Neuroguide on top of infrared therapy, resulting in another 50% improvement, on average, particularly in coherence and improvement in cognition.

It was also observed that similar results were obtained when combining rTMS with QEEG and biofeedback. Bernhard had implemented a low power, low frequency direct energy stimulation to the cortex with rTMS directed using four antenna directed at the back of the brain.

Putting rTMS energy into the cortex based on QEEG results is very different from using operand based feedback, as used by Berman, using the well-studied and reported Neuroguide methods, in which the patient watches television and real time analysis of QEEG results causes colorization or video responses which subliminally train brain function. This, in conjunction with Berman's combination only, showed great gains. Neuroguide software from Applied Neuroscience is the technology that looks at the brain, in real time, and records brain activities across nodes and compares them to the functioning of normal, healthy minds in a database. While watching television or listening to music, the brain may be retrained to work more optimally. (Further details regarding this may be found in the following publication: “Normative EEG Databases and EEG Biofeedback”, J. Neurother. 2(4), 8-39. Thatcher, R. W., & John, E. R. (eds). (1977)).

In one case study, administration of a combined therapy to the inventor of the present disclosure for a period of one month returned the slow memory back to normal. Further, the weekly QEEG tests indicated that the standard deviations from normative database of brain activity had recovered during the period of the combined therapy involving application of electrical fields, then infrared fields (initially at 1070 and then later at 660 nm), and also regular Neuroguide sessions using operands to correct slow and fast waves towards the normative indicators.

In another case study, the use of infrared light at 660 nm daily, for 5 minutes of exposure on left frontal damaged area of the skull showed effectiveness against memory loss for thirty days, consistently. It may also be noted that infrared light based therapy actually causes red marrow to elute stem cells in the bone into the nearby area, such as the cortex where there has been damage, as taught by Uri Omon. Additionally, the providing of infrared energy into neuronal tissue results in neuronal regeneration. In particular, with neuromodulation there is a rapid increase in both nerve pads, dendrites and synapses, as taught by neurobiofeedback at Tel-Aviv University.

Further studies show that use of infrared light can both up-regulate and deregulate endogenous functions. For example, 660 nanometers provokes the body to take up more blood and heat, while a wavelength of 880 nanometers retards this process.

It has been observed that various treatments for neurological disorders exhibit some degree of effectiveness. It has also been observed that combining two treatments can be more effective than either treatment alone. The present disclosure proposes that each treatment addresses a part of a larger reparative process wherein multiple components are necessary to effectively restore or modify neurological function. If this is correct, then as already noted, combinational treatments would in some cases produce better results than a single type of treatment.

### CONCLUSION

The present invention is described herein with reference to block diagrams and functional illustrations of methods and apparatus to implement various aspects of the present invention. It is understood that each block of the block diagrams or operational illustration or function represented, and combinations of blocks in the block diagrams or operational or functional illustrations, may be implemented by automated apparatus, such as analog or digital hardware and computer program instructions. These computer program instructions may be provided to a processor of a general purpose computer, special purpose computer, cellular device, smart device, ASIC, or other programmable data processing apparatus, such that the instructions, which execute via the processor of the computer or other programmable data processing apparatus, implements the functions/acts specified in the block diagrams or operational block or blocks. In some implementations, the functions or method steps described in relation to the blocks or functional representations may occur in an order other than the order noted or described herein, For example, blocks or functional representations shown in a succession may be executed substantially concurrently or the blocks in an alternate order, depending upon a specific implementation of the present invention. It is therefore understood that unless otherwise specifically noted and thereby limited, the discussion here is presented in an order to facilitate enablement and understanding and is not meant to limit the invention disclosed.

While this specification contains many specific implementation details, there should not be construed as limitations on the scope of any inventions or of what may be claimed, but rather as descriptions of features specific to particular embodiments of the present invention.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed invention.

What is claimed is:

1) Apparatus for providing trans-cranial directed energy therapy to a mammalian subject, the apparatus comprising:
a directed energy emission device positionable proximate to a surface of a cranium of the mammalian subject, said directed energy emission device having a plurality of energy-emitting portals;
a source of directed energy in communication with said plurality of energy-emitting portals and capable of emitting energy in a form of at least one of visible light, infrared light, near infrared light, electromagnetic fields, ultrasound, and millimeter waves directed towards the mammalian subject's cranium via the energy-emitting portals of the directed energy emission device;
a controller in logical communication with the source of directed energy and operative to cause the source of directed energy to provide a predetermined directed energy therapy to at least some of the energy portals, wherein the predetermined directed energy therapy comprises delivering at least one type of directed energy from a specific selection of energy portals for a predetermined period of time with a predetermined energy level, said controller comprising a processor and a digital data storage;
a biometric measurement device capable of quantifying one or more biological aspects of the mammalian subject, the biometric measurement device in logical communication with the controller at least one of constantly and intermittently to provide digital data to the controller indicative of the one or more measurements quantifying biological aspects of the mammalian subject at least one of before, during, and after an administration of the predetermined directed energy therapy, said digital data for use by at least one of: (i) a neurofeedback system in logical communication with the controller including external stimulation administered to the mammalian subject in the form of at least one of visual stimulation, audio stimulation, trans-cranial electrical stimulation, trans-cranial magnetic stimulation, trans-tongue electronical stimulation, and trans-dermal electrical stimulation; (ii) a biofeedback system in logical communication with the source of directed energy to at least one of continue and modify at least one of the predetermined directed energy therapy and neurofeedback administered to the mammalian subject based on the one or more measurements quantifying biological aspects of the mammalian subject; and (iii) a data storage device for storing the one or more measurements quantifying biological aspects from the mammalian subject at least one of before, during, and after at least one of the predetermined directed energy therapy and neurofeedback therapy administered to the mammalian subject; and
a source of electrical logic signals in logical communication with the controller and capable of causing the controller to receive input from the biometric measurement device and cause the source of directed energy to continue delivering the predetermined directed energy therapy or selectively modify the predetermined directed energy therapy to the mammalian subject based on the input from the biometric measurement device, wherein modifying the predetermined directed energy therapy includes at least one of modifying a selection of the energy portals, the type of directed energy, a level of directed energy, a duration of the directed energy, and a frequency of the directed energy.

2) apparatus of claim 1 wherein the controller is additionally in logical communication with a digital communications network and the source of electrical logic signals includes at least one network access device communicating the electrical logic signals via the digital communications network to deliver the predetermined directed energy therapy to the subject at a location remote from said controller.

3) apparatus of claim 2 wherein the network access device additionally receives and delivers the digital data indicative of feedback from the biometric measurement device to said controller at a location remote from said subject.

4) apparatus of claim 1 wherein the energy portals comprise at least one of: (a) a plurality of light emitting diodes capable of emitting light with a wavelength of between 450 nanometers and 1500 nanometers; (b) a plurality of fiber optic transmission elements each having a distal end for providing luminous communication from a source of light with a wavelength of between 450 nanometers and 1500 nanometers to the respective energy emission portal; (c) one or more ultrasound transducers; (d) one or more millimeter wave transducers; (e) one or more electrodes for delivering at least one of pulsed direct current (DC) and alternating current (AC); (f) one or more electromagnetic coils capable of emitting at least one of pulsed and static electromagnetic fields; and (g) combinational transducers capable of delivering any combination of types of directed energy.

5) apparatus of claim 1 wherein the multiple energy portals comprise a matrix and at least some of the matrix of energy portals may be activated by the controller to emit directed energy independent of other energy portals.

6) apparatus of claim 5 wherein multiple energy portals comprising the matrix are identifiable via predetermined coordinates selected from one or more coordinate types including Cartesian, Polar, number line, cylindrical, spherical, homogenous, curvilinear, orthogonal, skew log-polar, Plucker, canonical, parallel, barycentric, trilinear, and any transformation thereof, and wherein the controller may activate one or more of the energy portals according to a pattern that may be associated with multiple predetermined coordinates.

7) apparatus of claim 6 wherein the controller activates the source of directed energy to temperospatially deliver directed energy via emitting portals positioned proximate to one or both of a frontal lobe and a parietal lobe, and wherein the biometric measurement device provides feedback indicative of at least one of stimulation of and reduced activity in one or both of the frontal lobe and the parietal lobe.

8) apparatus of claim 7 wherein the controller at least one of: (a) identifies a front portion and a back portion of the matrix and activates energy portals in a pattern generally beginning at least one of at the front portion and back portion of the matrix and continuing to at least one of the back portion and front portion, respectively, of the matrix; (b) identifies a left portion and a right portion of the matrix and activates energy portals in a pattern generally beginning with at least one of the left portion and the right portion of the matrix and continuing to at least the right portion and left portion, respectively, of the matrix; (c) identifies a center portion and a periphery portion of the matrix and activates energy portals in a pattern generally beginning at least one of at the center portion and periphery of the matrix and continuing to at least one of the periphery portion and center portion, respectively, of the matrix; and (d) activates energy portals in a stochastic pattern throughout the matrix.

9) apparatus of claim 1 wherein the biometric measurement device comprises an electronic gaming device requiring inputs based upon cognitive analysis and manipulation of a user input mechanism.

10) apparatus of claim 1 wherein the biometric measurement device comprises at least one of a quantitative electroencephalography (QEEG) apparatus, an electromyography (EMG) apparatus, a thermometer, an electrodermography (EDG) apparatus, a photoplethysmography (PPG) apparatus, an electrocardiogram (ECG) apparatus, a pneumography apparatus, a capnography apparatus, a rheonocephalography (REG) apparatus, and a hemoencephalography (HEG) apparatus.

11) apparatus of claim 1, wherein said predetermined directed energy therapy is capable of regulating a production of adenosine triphosphate (ATP) and is selected to at least one of up regulate and down regulate the production of ATP of cells within the cranium of the subject.

12) apparatus of claim 11, wherein the predetermined directed energy to up regulate ATP production is selected having a peak wavelength of at least one of approximately 620 nanometers, approximately 680 nanometers, approximately 760 nanometers, and 820 nanometers.

13) apparatus of claim 11, wherein the predetermined directed energy to down regulate ATP production is selected having a peak wavelength of at least one of approximately 750 nanometers, 870 nanometers, 900 nanometers, and 950 nanometers.

14) apparatus of claim 1, wherein said predetermined directed energy therapy is capable of regulating at least one of an efficacy and a potency of at least one of pharmaceuticals, over-the-counter supplements, and medications to be administered to or by the subject and is selected to at least one of up regulate and down regulate at least one of the efficacy and the potency of at least one of pharmaceuticals, over-the-counter supplements, and medications to be administered to or by the subject.

15) apparatus of claim 1, wherein said predetermined directed energy therapy is capable of regulating at least one of natural DNA sequences, synthetic DNA sequences, and associated genes, sub-sets and/or pre-cursors of said natural or synthetic DNA sequences and is selected to at least one of up regulate and down regulate at least one of natural DNA sequences, synthetic DNA sequences, and associated genes, sub-sets and/or pre-cursors of said natural or synthetic DNA sequences.

16) apparatus of claim 1, wherein said predetermined directed energy therapy is at least one of selected: (a) from a pre-existing list of potential directed energy therapies based on a pre-therapy assessment of said subject with reference to an aggregated database incorporating at least one of pre-therapy assessments of other subjects, directed energy therapies of other subjects, and results of the directed energy therapies of other subjects; and (b) by a user of said apparatus based on a pre-therapy assessment of said subject at least one of with and without reference to said aggregated database.

17) apparatus of claim 16, wherein said pre-therapy assessment includes at least one of positron emission tomography (PET), computed tomography (CT), single-photo emission computed tomography (SPECT), blood biomarker testing, and cognitive testing to establish a functional baseline for the subject from which to compare the results of the predetermined directed energy therapy as determined by the biometric measurement device at least one of during and after the administration of the predetermined directed energy therapy.

18) apparatus of claim 1, wherein an effectiveness of said directed energy therapy may be augmented by at least one of physical exercise and mental exercise at least one of before, during and after said directed energy therapy.

19) apparatus of claim 1, wherein the directed energy emission device, the source of directed energy, the controller and the biometric measurement device of claim 1 are all part of a clinical directed energy system located in a first location, and further including a remote directed energy system located at a second location remote from the first location and including a second directed energy emission device, a second source of directed energy, a second controller, and a second biometric measurement device to collectively deliver the predetermined directed energy therapy as originated by the controller of the clinical directed energy system to the subject using the remote directed energy system.

20) apparatus of claim 19, wherein said predetermined directed energy therapy is capable of regulating at least one of an efficacy and a potency of at least one of pharmaceuticals, over-the-counter supplements, and medications to be administered to or by the subject and is selected to at least one of up and down regulate at least one of the efficacy and the potency of at least one of pharmaceuticals, over-the-counter supplements, and medications to be administered to or by the subject, and wherein the clinical directed energy system is capable of: (a) accessing electronic health records of the subject before an application of the predetermined directed energy therapy; (b) calculating an overall quanta of energy of the predetermined directed energy therapy to be administered to the subject; (c) calculating a degree of modification to an amount of pharmaceutical, over-the-counter supplement and/or medication to be administered to the subject based on an increase or a decrease in efficacy or potency due to the up or down regulation as a result of the predetermined directed energy therapy; and (d) communicating the degree of modification to at least one of a prescribing healthcare professional, a pharmacy associated with the subject, and the remote directed energy system.
